(19) United States
(12) Patent Application Publication
(10) Pub. No.: US 2008/0255093 A1
(43) Pub. Date: Oct. 16, 2008

(54) COMPOSITIONS AND METHODS FOR TREATING OBESITY AND RELATED DISORDERS

(76) Inventors: Peter Y. Tam, Redwood City, CA (US); Leland F. Wilson, Menlo Park, CA (US)

Correspondence Address: MINTZ, LEVIN, COHN, FERRIS, GLOVSKY AND POPEO, P.C, 1400 PAGE MILL ROAD, PALO ALTO, CA, 94304-1124, US

(21) Appl. No.: 12/111,793

(22) Filed: Apr. 29, 2008

Related U.S. Application Data
(63) Continuation-in-part of application No. 11/764,116, filed on Jun. 15, 2007.
Application No. 11/764,116 is a continuation-in-part of application No. 11/385,233, filed on Mar. 20, 2006.
Application No. 11/385,233 is a continuation-in-part of application No. 10/454,368, filed on Jun. 3, 2003, now Pat. No. 7,056,890.
Application No. 10/454,368 is a continuation-in-part of application No. 09/593,555, filed on Jun. 14, 2000, now abandoned.
(60) Provisional application No. 60/854,756, filed on Oct. 27, 2006.
(60) Provisional application No. 60/139,022, filed on Jun. 14, 1999.
(60) Provisional application No. 60/178,563, filed on Jan. 26, 2000.
(60) Provisional application No. 60/181,265, filed on Feb. 9, 2000.

(51) Int. Cl. A61K 31/55; A61K 31/137; A61K 31/35; A61P 3/04; A61P 25/00; A61P 1/16; A61P 9/12; A61P 3/10; A61K 31/42; A61K 31/454

(52) U.S. Cl. 514/217.01; 514/655; 514/455; 514/397; 514/326

(57) ABSTRACT

The present invention is drawn to combinations of pharmaceutical agents having similar chemical and/or pharmacological properties, wherein the combinations maximize the therapeutic effect of the drug while minimizing their adverse effects. The methods and compositions of the invention are particularly useful in the treatment of obesity and related conditions which involves treating a subject with a sympathomimetic agent (e.g., phentermine or a phentermine-like drug) or bupropion in combination with an anti-epileptic agent (e.g., topiramate, zonisamide), CB1 antagonists (e.g., rimonabant), or a 5HT_{2C}-selective serotonin receptor agonist, (e.g., lorcaserin) for the treatment of obesity and related conditions. The invention also features kits for use in the practice of these novel therapies.

### CROSS-REFERENCE TO RELATED APPLICATIONS

This application is a continuation-in-part of U.S. patent application Ser. No. 11/764,116, filed on Jun. 15, 2007, which claims priority to U.S. Patent Application Ser. No. 60/854,756, filed Oct. 27, 2006, and is a continuation-in-part of U.S. patent application Ser. No. 11/385,233, filed Mar. 20, 2006, which is a continuation-in-part of U.S. patent application Ser. No. 10/454,368, filed Jun. 3, 2003, now U.S. Pat. No. 7,056,890, which is a continuation-in-part of U.S. patent application Ser. No. 09/593,555, filed Jun. 14, 2000, now abandoned, which claims priority under 35 U.S.C. §119(e) (1) to provisional U.S. Patent Application Ser. No. 60/139,022, filed Jun. 14, 1999, Ser. No. 60/178,563, filed Jan. 26, 2000, and Ser. No. 60/181,265, filed Feb. 9, 2000. The aforementioned patent applications are incorporated herein by reference in their entireties.

### TECHNICAL FIELD

The invention relates generally to pharmaceutical compositions and methods for the treatment of various conditions, disorders, and diseases, and more particularly relates to the treatment of such conditions, disorders, and diseases using therapeutic agents that in combination provide advantages relative to the administration of either agent in a monotherapeutic regimen. The methods and compositions of the invention are particularly useful in the treatment of obesity and related conditions.

### BACKGROUND OF THE INVENTION

Many conditions, disorders, and diseases are treated with pharmaceutical agents, often on a regular basis for an extended period of time. For example, many pharmaceutical agents are commonly prescribed in the context of a continuing dosage regimen for the treatment of diabetes, hypertension, migraines, epilepsy, sleep apnea, depression, impulse control disorders, and alcohol addiction. Individuals who are overweight or obese are also commonly treated with therapeutic agents on an ongoing basis. While society has seen tremendous advances in the field of pharmaceuticals, there are, of course, drawbacks to the administration of any given pharmaceutical agent. Sometimes, the disadvantages, characterized as “side effects,” are so severe as to preclude administration of a particular agent at a therapeutically effective dose. In such a case, drug therapy is discontinued, and other pharmaceutical agents may be tried. Many agents in the same therapeutic class, however, display similar side effect profiles, meaning that patients either have to forego therapy or suffer from unpleasant side effects associated with a particular medication.

In addition, not everyone reacts the same way to the same medication. One person may experience an adverse reaction to a certain drug, while another person may have no problems at all. In another instance, a particular drug may improve a first patient's condition but offer no improvement to a second patient suffering from the same condition.

Accordingly, there is interest in the development of additional methods, compositions and dosing strategies for treating obesity and related conditions in which the therapeutic efficacy of known compositions are improved. Further, combination treatment may be employed to decrease the doses of the individual components in the resulting combinations while still preventing unwanted or harmful side effects of the individual components. Moreover, combination treatment offers a choice of various drugs for treating obesity or a related condition. As such, when one drug combination does not work in a particular individual, another combination may be administered which will be effective for treating obesity or a related condition. Thus, there is an urgent need to discover suitable methods for the treatment of obesity and related conditions, including combination treatments that result in reduction of toxicity, decreased side effects and effective.

The present invention is directed to the use of pharmaceutical agent combinations in which the side effects associated with one or both of the agents administered are reduced. By “indirectly” reducing side effects is meant that a first pharmaceutical agent allows the second agent to be administered at a lower dose without compromising therapeutic efficacy, thus resulting dose-dependent unwanted effects. While the invention is useful in conjunction with numerous pharmaceutical agents and therapeutic regimens, conditions of particular interest that may be treated according to the present methodology include obesity and related conditions such as those often associated and/or caused by obesity.

Overweight and obesity are widespread, serious problems in first world countries, especially in the United States, as well as in many developing countries such as China and India. Much money has been invested by both companies providing weight loss programs and products, and patients attempting to lose weight. Almost all aspects of a person are affected by overweight and obesity, from physical problems such as knee and ankle joint deterioration, to emotional problems due to society's rejection of overweight individuals. The medical problems caused by overweight and obesity can be serious and often life-threatening and include diabetes, shortness of breath and other respiratory problems, gallbladder disease, hypertension, dyslipidemia (for example, high cholesterol or high levels of triglycerides), cancer, osteoarthritis, other orthopedic problems, reflux esophagitis (heartburn), snoring, sleep apnea, menstrual irregularities, infertility, gout, problems associated with pregnancy, heart trouble, muscular dystrophy and metabolic disorders, including hypoalphalipoproteinemia, familial combined hyperlipidemia, insulin resistant syndrome X or multiple metabolic disorder, coronary artery disease, and dyslipidemic hypertension. In addition, obesity has been associated with an increased incidence of certain cancers, notably cancers of the colon, rectum, prostate, breast, uterus, and cervix.

Moreover, patients who are obese or overweight have a substantially increase risk of morbidity from hypertension, dyslipidemia, type 2 diabetes, coronary heart disease, stroke, gallbladder disease, osteoarthritis and endometrial, breast, prostate, and colon cancers. Higher body weights are also associated with increases in all-cause mortality. Most or all of these problems are relieved or improved by permanent significant weight loss as well as a significant increase in longevity.

The currently available strategies for treating these disorders include dietary restriction, increments in physical activity, pharmacological and surgical approaches which vary depending, at least in part, on the degree of weight loss one is attempting to achieve in a subject as well as on the severity of overweight or obesity exhibited by the subject. For example, treatments such as low-fat diet and/or regular exercise are often adequate in cases where a subject is only mildly overweight. Such treatments can be enhanced by controlled use of over-the-counter appetite suppressants including caffeine, ephedrine and phenylpropanolamine (Acutrim®, Dexatrim®). Moreover, prescription medications including amphetamine, diethylpropion (Tenuate®), mazindol (Mazanor®, Sanorex®), phentermine (Fastin®, Ionamin®), phenmetrazine (Preludin®), phendimetrazine (Bontrol®, Plegine®, Adipost®, Dital®, Dyrexan®, Melflat®, Prelu-2®, Rexigen Forte®), benzphetamine (Didrex®) and fluoxetine (Prozac®) are often used in the treatment of seriously overweight and/or obese subjects or patients. However, such treatments, at best, result in only 5˜10% weight loss (when accompanied with diet and exercise). Moreover, most of these treatments ultimately prove inadequate because they are either dangerous, ineffective or quickly lose their anorexient effect. In adults, long term weight loss is exceptional using conservative interventions. Present pharmacological interventions typically induce a weight loss of between five and fifteen kilograms; if the medication is discontinued, renewed weight gain ensues. Surgical treatments are comparatively successful and are reserved for patients with extreme obesity and/or with serious medical complications.

Available Drug Therapies:

Various combination therapies that include the sympathomimetic agent phentermine have been investigated and have met with mixed success. The phentermines were, until around 1997, often prescribed along with fenfluramine (Pondimin®) or dexfenfluramine (Redux®), nicknamed “fen”, as a combination therapy known as fen-phen. Fenfluramine is a potent releaser of serotonin from serotonergic neurons which acts on a cerebral appetite center. When combined with phentermine, fenfluramine had the effect of enhancing and extending the anorexient action of phentermine. However in 1997, the Food and Drug Administration (“FDA”) asked manufacturers to withdraw Pondimin® and Redux® due to studies which strongly suggested that the drugs cause damage to the mitral valve of the heart and pulmonary hypertension.

More recently, it has been suggested that phentermine in combination with an anti-depressant agent is a potentially effective therapy for effecting weight loss (U.S. Pat. No. 5,795,895). In particular, the anti-depressants suggested for use in this new combination therapy are members of a class of compounds known as selective serotonin reuptake inhibitors (SSRIs) which include fluoxetine (Prozac®), sertraline (Zoloft®), fluvoxamine maleate (Luvox®) and trazodone hydrochloride (Desyrel®). The combination therapy is also suggested to treat coexisting depression and/or obsessive-compulsive disorder.

Phentermine has also recently been tested in combination with bupropion (Wellbutrin®) for the treatment of obesity. (Bradley et al. (1999) “Bupropion SR with Phentermine for Weight Reduction,” Books of Abstracts, American Psychiatric Association Meeting (distributed to meeting attendees), Washington, D.C. (abstract only)). Bupropion is an antidepressant that inhibits dopamine reuptake, as compared to serotonin uptake. It is also used to treat attention deficit disorders such as Attention Deficit Hyperactivity Disorder (ADHD), bipolar depression, chronic fatigue syndrome, cocaine addiction, nicotine addiction, and lower back pain. While bupropion alone had a modest effect as a weight loss agent (when prescribed to patients following a 1200 calorie per day diet), patients receiving phentermine in combination with bupropion experienced no greater weight loss than those receiving bupropion alone. Moreover, bupropion use has been associated with medication induced seizures causing it to be removed from the market by the FDA for at least five years before its re-introduction in 1989.

Zonisamide (ZONEGRAN™), a sulfonamide antiepileptic drug, is used to control some kinds of seizures in the treatment of epilepsy. Zonisamide may produce these effects through action at sodium and calcium channels. In vitro pharmacological studies suggest that zonisamide blocks sodium channels and reduces voltage-dependent, transient inward currents (T-type Ca²⁺ currents), consequently stabilizing neuronal membranes and suppressing neuronal hypersynchronization. In vitro binding studies have demonstrated that zonisamide binds to the GABA/benzodiazepine receptor ionophore complex in an allosteric fashion which does not produce changes in chloride flux. Other in vitro studies have demonstrated that zonisamide (10-μg/mL) suppresses synaptically-driven electrical activity without affecting postsynaptic GABA or glutamate responses (cultured mouse spinal cord neurons) or neuronal or glial uptake of [³H]-GABA (rat hippocampal slices). Thus, zonisamide does not appear to potentiate the synaptic activity of GABA. In vivo microdialysis studies demonstrated that zonisamide facilitates both dopaminergic and serotonergic neurotransmission. Zonisamide also has weak carbonic anhydrase inhibiting activity, but this pharmacologic effect is not thought to be a major contributing factor in the antiseizure activity of zonisamide. Side effects of zonisamide include but are not limited to: renal calculi, drowsiness, ataxia, loss of appetite, gastrointestinal symptoms, severe rash (i.e. Stevens Johnson Syndrome [SJS] and toxic epidermal necrolysis [TEN]), serious hematologic events, such as aplastic anemia or agranuclocytosis, oligohydrosis and hyperthermia in pediatric patients.

2,3,4,5-Bis-O-(1-methylethylidene)-β-D-fructopyranose sulfamate, known as topiramate, is a member of a class of antiepileptic drugs and has been demonstrated in clinical trials of human epilepsy to be effective as adjunctive therapy or as monotherapy in treating simple and complex partial seizures and secondarily generalized seizures (E. Faught et al. (1996) Neurology 46:1684-90.; Karim et al. (1995) Epilepsia 36 (S4):33; S. K. Sachdeo et al. (1995) Epilepsia 36(S4):33; T. A. Glauser (1999) Epilepsia 40 (S5):S71-80; R. C. Sachdeo (1998) Clin. Pharmacokinet. 34:335-346), and is currently marketed for the treatment of seizures in patients with simple and complex partial epilepsy and seizures in patients with primary or secondary generalized seizures in the United States, Europe and most other markets throughout the world. There has also been evidence that topiramate is effective in the treatment of diabetes (U.S. Pat. Nos. 7,109,174 and 6,362,220), neurological disorders (U.S. Pat. No. 6,908,902), depression (U.S. Pat. No. 6,627,653), psychosis (U.S. Pat. No. 6,620,819), headaches (U.S. Pat. No. 6,319,903) and hypertension (U.S. Pat. No. 6,201,010). However there have been adverse effects associated with the use of topiramate in humans.

It has been found that CB1 receptor antagonists may be used in the treatment of obesity. Recent studies have demonstrated that activation of CB1 receptors by endogenous cannabinoids, such as anadamide, may cause increases in appetite. Therefore, CB1 antagonists or inverse agonists are currently under investigation for controlling appetite in treating obese or overweight patients. For example, rimonabant is a CB1 cannabinoid receptor antagonist which causes a significant reduction in appetite. (Makriyannis et al. (2005), Neuro Pharma 48:1068-1071) The medication causes, among other effects, decrease in appetite, possible increase in metabolic activity, and blockage of lipogenesis. See, e.g., Despres et al. (2005) NEJM 353: 2121-34.

However, rimonabant did not enter the market in the United States because the FDA required additional information pertaining to associations between rimonabant and increased rates of psychiatric adverse events, including depression and suicidality and neurological adverse events, including seizures. (FDA: MEMORANDUM from Division of Metabolism and Endocrinology Products (May 22, 2007)) As such, the use of CB1 antagonists for the treatment of obesity has been hindered by concomitant side effects such as sedation and depression of the CNS. Thus, a potential treatment for obesity is marred by the negative effects a patient would need to endure in order to lose weight—a major problem for patient compliance and for the patient's health.

These drugs and others have shown some promise as therapeutic agents in treating the above-mentioned pathologies. However, as noted earlier herein, many if not all of these drugs have adverse reactions that limit their ability to be used in humans at therapeutically effective doses. The following embodiments of the instant invention are drawn to combinations of pharmaceutical agents such as the drugs mentioned hereinabove and other drugs having similar chemical and/or pharmacological properties, wherein the combinations maximize the therapeutic effect of the drug while minimizing their adverse effects.

### SUMMARY OF THE INVENTION

The invention is directed to compositions and methods wherein two or more therapeutic agents are used in combination and administered for the treatment of obesity and related conditions. The invention involves administering a combination of therapeutic agents wherein a second agent directly or indirectly reduces the unwanted side effects resulting from administration of the first agent. Generally, although not necessarily, the methodology of the invention is implemented in the context of a prolonged dosage regimen, e.g., involving daily or weekly dosing for a period of weeks or even months or years. By combining the agents according to the present invention, each reduces the side effects of the other agent and both contribute to the pharmacology effect of enhancing weight loss. Because they both enhance weight loss, one can reduce the dose of each component to make the combination more tolerable.

In certain aspects, the drug combinations of the present invention provide a choice of various drugs for an individual who does not react to one particular combination or experiences adverse side effects. As such, a second combination of drugs may be administered to an individual when a first drug combination was not effective for treating obesity or a related condition.

The present invention, in one embodiment, features a novel therapy for treating obesity and related conditions, including conditions associated with and/or caused by obesity per se. The method involves treating a subject with a first pharmaceutical agent and a second pharmaceutical agent, wherein the second pharmaceutical agent is a sympathomimetic agent. The term sympathomimetic agent is a term of art and refers to agents or compounds which mimic or alter stimulation of the sympathetic nervous system.

In certain embodiments, the present invention is directed towards a composition for treating obesity or a related condition in a subject which includes a first pharmaceutical agent and a second pharmaceutical agent, wherein the second pharmaceutical agent is a sympathomimetic agent and wherein the first pharmaceutical agent is an anti-epileptic agent, CB1 receptor antagonist, or a 5HT_{2C}-selective serotonin receptor agonist.

In certain aspects of the present invention, the sympathomimetic agent is phentermine or bupropion.

In certain embodiments of the present invention, the first pharmaceutical agent is an anti-epileptic agent.

In certain aspects of the present invention, the anti-epileptic agent is selected from the group consisting of topiramate, zonisamide, γ-vinyl GABA (vigabatrin), carbamazepine, clonazepam, ethosuximide, gabapentin, lamotrigine, levetiracetam, phenobarbital, phenyloin, primidone, tiagabine, valproate, felbamate, oxazinane-dione, metharbital, ethotoin and mesantoin, vigabatrin, gabapentin, and oxcarbazepine.

In certain aspects, the anti-epileptic agent is topiramate.

In certain aspects, the anti-epileptic agent is zonisamide.

In certain embodiments of the present invention, the first pharmaceutical agent is a CB1 receptor antagonist.

In certain aspects, CB1 receptor antagonist is selected from the group consisting of rimonabant, SLV-326, SLV-319, AM251, AM4113, AM281, Taranabant, NIDA-41020, NEWW 0327, O-2050, O-2654, CP-272871, CP-945598, CP-946598, AVE1625, Surinabant, LY320135, AVN-342, GRC-10389, Org-50189, PSNCBAM-1, E-6776, V-24343, ACPA, ACEA, HU-210, and HU-243.

In certain aspects, the CB1 receptor antagonist is rimonabant.

In certain embodiments of the present invention, the first pharmaceutical agent is a 5HT_{2C}-selective serotonin receptor agonist.

In certain aspects, the 5HT_{2C}-selective serotonin receptor agonist is selected from the group consisting of lorcaserin, mesulergine, agomelatine, fluoxetine, BVT933, DPCA37215, 1K264; PNU 22394; WAY161503, R-1065, and YM 348.

In certain aspects, the 5HT_{2C}-selective serotonin receptor agonist is lorcaserin.

In certain embodiments, the present invention is directed to a dosage form of a pharmaceutical composition comprising a combination of an immediate release form of the sympathomimetic agent and a controlled release form of the anti-epileptic agent, CB1 receptor antagonist, or 5HT_{2C}-selective serotonin receptor agonist.

In certain aspects, the dosage form comprises an immediate release form of phentermine while in other aspects, the dosage form comprises an immediate release form of bupropion.

In certain aspects, the dosage form comprises a controlled release form of an anti-epileptic agent. In certain aspects, the anti-epileptic agent is topiramate, while in other aspects, the anti-epileptic agent is zonisamide.

In certain aspects, the dosage form comprises a controlled release form of the CB1 receptor antagonist. In certain aspects, the CB1 receptor antagonist is rimonabant.

In certain aspects, the dosage form comprises a controlled release form of the 5HT_{2C}-selective serotonin receptor agonist. In certain aspects, the 5HT_{2C}-selective serotonin receptor agonist is lorcaserin.

In some embodiments, the present invention is directed towards a method for treating obesity or a related condition in a subject comprising administering to the subject a first pharmaceutical agent and a second pharmaceutical agent, wherein the second pharmaceutical agent is a sympathomimetic agent and wherein the first pharmaceutical agent is an anti-epileptic agent, CB1 receptor antagonist, or a 5HT_{2C}-selective serotonin receptor agonist.

In certain aspects, the method includes administering the first pharmaceutical agent and the second pharmaceutical agent separately.

In certain aspects, the method includes administering the first pharmaceutical agent and the second pharmaceutical agent at different times of the day.

In certain aspects, the method includes administering the second pharmaceutical agent in the morning and administering the first pharmaceutical agent at least once later in the day.

In certain aspects, the related condition is pre-diabetes, insulin-resistance or diabetes.

In certain aspects, the related condition is hypertension.

In certain aspects, the related condition is sleep apnea.

In certain aspects, the related condition is nonalcoholic steatohepatitis.

In certain aspects, the related condition is nonalcoholic fatty liver disease.

In certain aspects, the related condition is diabetic nephropathy.

In some embodiments, the present invention is directed towards a kit comprising a packaged combination of a first pharmaceutical agent and a second pharmaceutical agent, wherein the second pharmaceutical agent is a sympathomimetic agent and wherein the first pharmaceutical agent is an anti-epileptic agent, CB1 receptor antagonist, or a 5HT_{2C}-selective serotonin receptor agonist and instructions for a patient to carry out drug administration to achieve weight loss, wherein the first and second pharmaceutical agents are present in separate and discrete dosage forms.

In some embodiments, the present invention is directed towards a kit comprising a sealed package of controlled release dosage forms each containing a first pharmaceutical agent and a second pharmaceutical agent, wherein the second pharmaceutical agent is a sympathomimetic agent and wherein the first pharmaceutical agent is an anti-epileptic agent, CB1 receptor antagonist, or a 5HT_{2C}-selective serotonin receptor agonist, wherein the dosage forms provide for immediate release of the second pharmaceutical agent and delayed release of the first pharmaceutical agent.

The present invention also features a pharmaceutical composition that includes, e.g., bupropion in combination with an anti-epileptic agent, a CB1 antagonist, or a 5HT_{2C}-selective serotonin receptor agonist. The present invention additionally features a pharmaceutical composition that includes, e.g., phentermine in combination with an anti-epileptic agent, a CB1 antagonist, or a 5HT_{2C}-selective serotonin receptor agonist. For example, the pharmaceutical composition contains topiramate or zonisamide or rimonabant in combination with phentermine or bupropion.

The present invention also features a pharmaceutical composition that includes topirimate in combination with phentermine for treating obesity or a related condition.

The present invention also features a pharmaceutical composition that includes zonisamide in combination with phentermine for treating obesity or a related condition.

The present invention also features a pharmaceutical composition that includes rimonabant in combination with phentermine for treating obesity or a related condition.

The present invention also features a pharmaceutical composition that includes lorcaserin in combination with phentermine for treating obesity or a related condition.

The present invention also features a pharmaceutical composition that includes topirimate in combination with bupropion for treating obesity or a related condition.

The present invention also features a pharmaceutical composition that includes zonisamide in combination with bupropion for treating obesity or a related condition.

The present invention also features a pharmaceutical composition that includes rimonabant in combination with bupropion for treating obesity or a related condition.

The present invention also features a pharmaceutical composition that includes lorcaserin in combination with bupropion for treating obesity or a related condition.

### DETAILED DESCRIPTION OF THE INVENTION

Before describing the present invention in detail, it is to be understood that unless otherwise indicated, this invention is not limited to particular formulations, active and inactive agents, modes of administration, or methods of treatment or use, as such may vary. It is also to be understood that the terminology used herein is for the purpose of describing particular embodiments only, and is not intended to be limiting. The scientific publications, patents or patent applications cited in the various sections of this document are herein incorporated-by-reference for all purposes.

### DEFINITIONS AND NOMENCLATURE

It must be noted that, as used in this specification and the appended claims, the singular forms “a,” “an” and “the” include plural referents unless the context clearly dictates otherwise. Thus, for example, “an active agent” refers not only to a single active agent but also to a combination of two or more different active agents, “a dosage form” refers to a combination of dosage forms as well as to a single dosage form, and the like. Unless defined otherwise, all technical and scientific terms used herein have the meaning commonly understood by one of ordinary skill in the art to which the invention pertains. Although any methods and materials similar or equivalent to those described herein may be useful in the practice or testing of the present invention, preferred methods and materials are described below. Specific terminology of particular importance to the description of the present invention is defined below.

When referring to an active agent, applicants intend the term “active agent” to encompass not only the specified molecular entity but also its pharmaceutically acceptable, pharmacologically active analogs, including, but not limited to, salts, esters, amides, prodrugs, conjugates, active metabolites, and other such derivatives, analogs, and related compounds.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, “treating” a patient as described herein encompasses treating obesity or a related condition in an individual.

By the terms “effective amount” and “therapeutically effective amount” of an agent, compound, drug, composition or combination of the invention which is nontoxic and effective for producing some desired therapeutic effect upon administration to a subject or patient (e.g., a human subject or patient).

The term “dosage form” denotes any form of a pharmaceutical composition that contains an amount of active agent sufficient to achieve a therapeutic effect with a single administration. When the formulation is a tablet or capsule, the dosage form is usually one such tablet or capsule. The frequency of administration that will provide the most effective results in an efficient manner without overdosing will vary with the characteristics of the particular active agent, including both its pharmacological characteristics and its physical characteristics, such as hydrophilicity.

The term “controlled release” refers to a drug-containing formulation or fraction thereof in which release of the drug is not immediate, i.e., with a “controlled release” formulation, administration does not result in immediate release of the drug into an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein includes sustained release and delayed release formulations.

The term “sustained release” (synonymous with “extended release”) is used in its conventional sense to refer to a drug formulation that provides for gradual release of a drug over an extended period of time, and that preferably, although not necessarily, results in substantially constant blood levels of a drug over an extended time period. The term “delayed release” is also used in its conventional sense, to refer to a drug formulation which, following administration to a patient, provides a measurable time delay before drug is released from the formulation into the patient's body.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” derivative or analog, refers to a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

### Active Agent Combinations:

The instant invention provides for the pharmaceutical treatment of many conditions, disorders, and diseases wherein side effects are significantly reduced. For instance, the invention provides for the treatment of obesity and related conditions associated with and/or caused by obesity, e.g., diabetes, hypertension, and sleep apnea, depression. The subject invention involves treating a subject with a first pharmaceutical agent and a second pharmaceutical agent, wherein the first pharmaceutical agent is an anti-epileptic agent, CB1 receptor antagonist or a 5HT_{2C}-selective serotonin receptor agonist and the second pharmaceutical agent is a sympathomimetic agent. By combining the agents according to the present invention, each reduces the side effects of the other agent and both contribute to the pharmacology effect of enhancing weight loss. Because they both enhance weight loss, one can reduce the dose of each component to make the combination more tolerable.

Sympathomimetic agents for use in the present invention and their general clinical uses or effects are set forth in Table I.

TABLE I
Sympathomimetic Agents and Clinical Uses Thereof

i

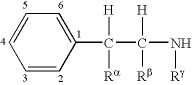

| General structure: | | | | | Main Clinical Uses | | |
|---|---|---|---|---|---|---|---|
| | Ring | | | | α Receptor | β Receptor | CNS, |
| Agent name | substituent(s) | R^{α} | R^{β} | R^{γ} | A N P V | B C | 0 |
| Bupropion | 3-Cl | ═O | CH₃ | C(CH₃)₃ | | | |
| Phenylethylamine | | H | H | H | | | |
| Epinephrine | 3-OH, 4-OH | OH | H | CH₃ | A, P, V | B, C | |
| Norepinephrine | 3-OH, 4-OH | OH | H | H | P | | |
| Epinine | 3-OH, 4-OH | H | H | CH₃ | | | |
| Dopamine | 3-OH, 4-OH | H | H | H | P | | |
| Dobutamine | 3-OH, 4-OH | H | H | I* | | C | |
| Nordefrin | 3-OH, 4-OH | OH | CH₃ | H | V | | |
| Ethylnorepinephrine | 3-OH, 4-OH | OH | CH₂CH₃ | H | | B | |
| Isoproterenol | 3-OH, 4-OH | OH | H | CH(CH₃)₂ | | B, C | |
| Protokylol | 3-OH, 4-OH | OH | H | 2* | | B | |
| Isoetharine | 3-OH, 4-OH | OH | CH₂CH₃ | CH(CH₃)₂ | | B | |
| Metaproterenol | 3-OH, 5-OH | OH | H | CH(CH₃)₂ | | B | |
| Terbutaline | 3-OH, 5-OH | OH | H | C(CH₃)₃ | | B | |
| Metaraminol | 3-OH | OH | CH₃ | H | P | | |
| Phenylephrine | 3-OH | OH | H | CH₃ | N, P | | |
| Tyramine | 4-OH | H | H | H | | | |
| Hydroxyamphetamine | 4-OH | H | CH₃ | H | N, P | C | |
| Methoxyphenamine | 2-OCH₃ | H | CH₃ | CH₃ | | B | |
| Methoxamine | 2-OCH₃, 5- | OH | CH₃ | H | P | | |
| | OCH₃ | | | | | | |
| Albuterol | 3-CH₂OH, 4- | OH | H | C(CH₃)₃ | | B | |
| | OH | | | | | | |
| Amphetamine | | H | CH₃ | H | | | CNS, |
| | | | | | | | 0 |
| Methamphetamine | | H | CH₃ | CH₃ | P | | CNS, |
| | | | | | | | 0 |
| Benzphetamine | | H | CH₃ | —NHR^{γ} is | | | 0 |
| | | | | replaced | | | |
| | | | | with 3* | | | |
| Ephedrine | | OH | CH₃ | CH₃ | N, P | B, C | |
| Phenylpropanolamine | | OH | CH₃ | H | N | | |
| Mephentermine | | H | —CHR^{β}— | CH₃ | N, P | | |
| | | | is replaced | | | | |
| | | | with 4* | | | | |
| Phentermine | | H | ″ | H | | | 0 |
| Chlorphentermine | 4-Cl | H | ″ | H | | | 0 |
| Fenfluramine | 3-CF₃ | H | CH₃ | C₂H₅ | | | 0 |
| Propylhexedrine | 5*: phenyl ring | H | CH₃ | CH₃ | N | | |
| | is replaced with | | | | | | |
| | cyclohexyl | | | | | | |

| | | |
|---|---|---|
| Diethylpropion | 6*: The substituent at the 1- | 0 |
| | position is replaced with 6, | |
| | below. | |
| Phenmetrazine | 7*: The substituent at the 1- | 0 |
| | position is replaced with 7, | |
| | below. | |
| Phendimetrazine | 8*: The substituent at the 1- | 0 |
| | position is replaced with 8, | |
| | below. | |

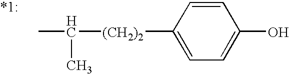

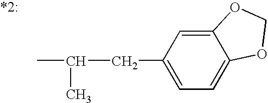

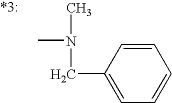

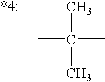

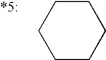

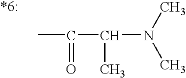

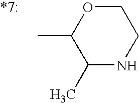

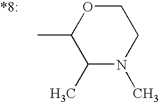

α Activity
A = Allergic reactions (includes β action)
N = Nasal decongestion
P = Pressor (may include β action)
V = Other local vasoconstriction (e.g. in local anesthesia)
β Activity
B = Bronchodilator
C = Cardiac
CNS = Central nervous system
0 = Anorectic
*Numbers bearing an asterisk refer to the substituents numbered in the bottom rows of the table; substituent 5 replaces the phenyl rings, and 6, 7 and 8 are attached directly to the phenyl ring, replacing the ethylamine side chain.
^{†}The α and β in the prototype formula refer to positions of the C atoms in the ethylamine side chain.

In certain embodiments, the sympathomimetic agent is phentermine or a phentermine-like compound. As defined herein, a phentermine-like compound is a compound structurally related to phentermine (e.g., an analog or derivative) which maintains an anorectic activity similar to that of phentermine. One phentermine-like compound is chlorphentermine. In yet another embodiment, the sympathomimetic agent is amphetamine or an amphetamine-like compound. As used herein, an amphetamine-like compound is a compound structurally related to amphetamine (e.g., an analog or derivative) which maintains an anorectic effect of amphetamine. In yet another embodiment, the sympathomimetic agent is phenmetrazine or a phenmetrazine-like compound. As defined herein, a phenmetrazine-like compound is a compound structurally related to phenmetrazine (e.g., an analog or derivative) which maintains an anorectic effect of phenmetrazine. One phenmetrazine-like compound is phendimetrazine. Analogs and/or derivatives of the compounds of the present invention can be tested for their ability to suppress appetite (e.g., suppress food intake) in a subject (e.g., a mammalian subject).

In other embodiments, the sympathomimetic agent is bupropion or a bupropion-like compound. As defined herein, a bupropion-like compound is a compound structurally related to bupropion (e.g., an analog or derivative) which maintains an anti-depressive activity similar to that of bupropion.

In an exemplary embodiment, the sympathomimetic agent is selected from bupropion, amphetamine, methamphetamine, benzphetamine, phenylpropanolamine, phentermine, chlorphentermine, diethylpropion, phenmetrazine, and phendimetrazine (as set forth in Table I). In one embodiment, the sympathomimetic agent is phentermine. It is also within the scope of the present invention to utilize other sympathomimetic agents including pseudo ephedrine (a stereoisomer of ephedrine, SUDAFED®), methylphenidate (RITALIN®), tuaminoheptane, other CNS stimulants including, for example, caffeine and bupropion.

In certain aspects of the invention, the first pharmaceutical agent is an anti-epileptic agent which are generally imidazoles (such as imidazole per se), imidazole derivatives, sulfonamides (such as topiramate), and sulfonylureas (such as zonisamide). Any anti-epileptic agents include GABA-T inhibitors like γ-vinyl GABA (vigabatrin). Anti-epileptic agents include carbamazepine, clonazepam, ethosuximide, gabapentin, lamotrigine, levetiracetam, phenobarbital, phenyloin, primidone, tiagabine, topiramate, valproate, felbamate, oxazinane-dione, metharbital, ethotoin and mesantoin, vigabatrin, gabapentin, and oxcarbazepine. Antiepileptic agents also include anticonvulsant sulfamate compounds and anticonvulsant sulfonylurea compounds as further defined below.

The terms anticonvulsant sulfamate compound (s), anticonvulsant sulfamate agent(s) anticonvulsant sulfamate derivative(s) or anticonvulsant sulfamate drug(s) are terms of art and refer to a class of sulfamate-derived compounds that possess anticonvulsant activity and have an art-recognized use in the treatment of epilepsy. In particular, the anticonvulsant sulfamate compounds are monosaccharide derivatives with sulfamate functionality. The anticonvulsant sulfamate compounds for use in the present invention have one or more of the following modes of activity: modulation of voltage-dependent sodium conductance; potentiation of gamma-aminobutyric acid-evoked currents; inhibition of the kainate/alpha-amino-3-hydroxy-5-methylisoxazole-4-propionic acid (AMPA) subtype of the glutamate receptor; and/or inhibition of carbonic anhydrase (e.g., a mechanism by which the anticonvulsant derivative of the present invention may decrease the sensation of taste). The anticonvulsant sulfamate compounds for use in the present invention are described further in U.S. Pat. Nos. 4,513,006, 5,384,327, 5,498,629, 5,753,693 and 5,753,694, as are methods of synthesizing such anticonvulsant sulfamate compounds. The aforementioned patents are incorporated by reference herein in their entireties.

In certain embodiments, the anticonvulsant sulfamate compound is a compound having the following formula (I):

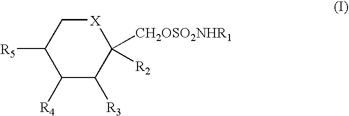

wherein:

X is CH₂ or O;

R₁ is H or alkyl; and

R₂, R₃, R₄ and R₅ are independently H or lower alkyl, with the proviso that when X is O, then R₂ and R₃ and/or R₄ and R₅ together may be a methylenedioxy group of the following formula (II):

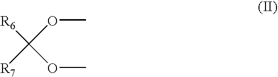

in which R₆ and R₇ are the same or different and are H or lower alkyl, or are joined to form a cyclopentyl or cyclohexyl ring.

R₁ in particular is hydrogen or alkyl of about 1 to 4 carbons, such as methyl, ethyl, or isopropyl. Alkyl includes both straight and branched chain alkyl. Alkyl groups R₂, R₃, R₄, R₅, R₆ and R₇ are about 1 to 3 carbons and include methyl, ethyl, isopropyl and n-propyl.

A particular group of compounds of the formula (I) are those wherein X is oxygen and both R₂ and R₃, and R₄ and R₅ together are methylenedioxy groups of the formula (II), wherein R₆ and R₇ are both hydrogen, both alkyl, or combine to form a spiro cyclopentyl or cyclohexyl ring, in particular, where R₆ and R₇ are both alkyl such as methyl. A second group of compounds are those wherein X is CH₂ and R₄ and R₅ are joined to form a benzene ring. A third group of compounds of the formula (I) are those wherein both R₂ and R₃ are hydrogen.

In one embodiment, the anticonvulsant sulfamate compound is topiramate (Topamax®). Topiramate, also referred to in the art as 2,3:4,5-bis-O-(1-methylethylidene)-β-D-fructopyranose sulfamate, has been demonstrated in clinical trials of human epilepsy to be effective as an adjunctive therapy or as monotherapy in treating simple and complex partial seizures and secondarily generalized seizures (E. Faught et al. (1995) Epilepsia 36(suppl 4):33; S. Sachdeo et al. (1995) Epilepsia 36(suppl 4):33) and is currently marketed for the treatment of simple and complex partial seizure epilepsy with or without secondary generalized seizures.

In another embodiment, the sulfamate compound is selected from 2,3-O-(1-methylethylidene)-4,5-O-sulfonyl-beta-D-fructopyranose sulfamate; 2,3-O-(1-methylethylidene)-4,5-O-sulfonyl-beta-L-fructopyranose sulfamate; 2,3-O-(1-methylethylidene)-4,5-O-sulfonyl-beta-D-fructopyranose methylsulfamate; 2,3-O-(1-methylethylidene)-4,5-O-sulfonyl-beta-D-fructopyranose butylsulfamate; 2,3-O-(1-methylethylidene)-4,5-O-sulfonyl-beta-D-fructopyranose ethylsulfamate; 2,3-O-(1-methylethylidene)-4,5-O-sulfonyl-beta-D-fructopyranose octylsulfamate; 2,3-O-(1-methylethylidene)-4,5-O-sulfonyl-beta-D-fructopyranose 2-propenylsulfamate; 2,3-O-(1-methylethylidene)-4,5-O-sulfonyl-beta-D-fructopyranose phenylmethylsulfamate; 2,3-O-(1-methylethylidene)-4,5-O-sulfonyl-beta-D-fructopyranose cyclopropylsulfamate; 2,3-O-(1-methylethylidene)-4,5-O-sulfonyl-beta-D-fructopyranose cyclobutylsulfamate; 2,3-O-(1-methylethylidene)-4,5-O-sulfonyl-beta-D-fructopyranose (2,2,2-trifluoroethyl)sulfamate; 2,3-O-(1-methylethylidene)-4,5-O-sulfonyl-beta-D-fructopyranose dimethylsulfamate; 2,3-O-(1-methylethylidene)-4,5-O-sulfonyl-beta-D-fructopyranose diethylsulfamate; 2,3-O-(1-methylethylidene)-4,5-O-sulfonyl-beta-D-fructopyranose azido sulfamate; (S)-2,3-O-(1-methylethylidene)-4,5-O-sulfinyl-beta-D-fructopyranose sulfamate; (R)-2,3-O-(1-methylethylidene)-4,5-O-sulfinyl-beta-D-fructopyranose sulfamate; 2,3-O-(1-ethylpropylidene)-4,5-O-sulfonyl-beta-D-fructopyranose sulfamate; 2,3-O-(1-methylethylidene)-4,5-O—[N-(4-methylbenzenesulfonyl)imidosulfonyl]-beta-D-fructopyranose sulfamate; 2,3-O-(1-methylethylidene)-4,5-O—[N-(4-methylbenzenesulfonyl)imidosulfonyl]-beta-D-fructopyranose sulfamate; 2,3-O-(cyclohexylidene)-4,5-O-sulfonyl-beta-D-fructopyranose sulfamate and (S)-4,5-O—[N-(1,1-dimethylethoxycarbonyl)imidosulfinyl]-2,3-O-(1-methylethy lidene)-beta-D-fructopyranose sulfamate.

In one embodiment the anticonvulsant sulfonylurea compound is zonisamide. Zonisamide (ZONEGRAN™), a sulfonylurea antiepileptic drug, is used to control some kinds of seizures in the treatment of epilepsy. Zonisamide may produce these effects through action at sodium and calcium channels. Other sulfonylurea antiepileptic drugs include chlorpropamide, tolazamide, tolbutamide, glyburide, glipizide and glimepiride.

In other embodiments of the invention, the first pharmaceutical agent is a CB1 receptor antagonist. Examples of a CB1 receptor antagonist or inverse agonist include rimonabant (SR141716A) (known in Europe as Acomplia® and supplied by Sanofi-Aventis); SLV-326, SLV-319 (Solvay); AM251, AM4113 and AM281 (Univ. of Conn.); Taranabant, i.e., MK-0364 and MK-0493 (Merck); NIDA-41020 (Sigma); NEWW 0327 (Sigma); O-2050 and O-2654 (Organix), CP-272871, CP-945598, CP-946598 (Pfizer), AVE1625, Surinabant, i.e., SR-14778 (Sanofi-Aventis), LY320135 (Lilly), AVN-342 (Azevan Pharmaceuticals), GRC-10389 (Glenmark), Org-50189 (Organon), PSNCBAM-1 (Prosidion), E-6776 (Esteve), V-24343 (Vemalis), the aryl-benzo[b]thiophene and benzo[b]furan compounds disclosed in U.S. Pat. No. 5,547,524, the N-{1-[bis(4-chlorophenyl)methyl]azetidin-3-yl}-N-(3,5-difluorophenyl)meth-ylsulfonamide, Diaryl-pyrazine-amide derivatives described in WO 03/051851, ACPA and ACEA from Med. Coll. Wisconsin (Univ. Aberdeen), (“Effects of AM 251 & AM 281, cannabinoid CB1 antagonists, on palatable food intake in lewis rats” J. Pharmacol. Exp. Ther. 289, No 3, 1427-33, 1999), Pyrazole derivatives described in the WO 01/29007, HU-210 (International Association for the Study of Pain—Ninth World Congress (Part II) Vienna, Austria, Dickenson A H, Carpenter K, Suzuki R, IDDB MEETING REPORT 1999, Aug. 22-27) and HU-243 (Cannabinoid receptor agonists and antagonists, Barth F, Current Opinion in Therapeutic Patents 1998, 8:3 (301-313)) from Yissum R&D Co Hebrew Univ. of Jerusalem, as well as the azetidine derivatives disclosed in WO 00/15609, WO 01/64633 and WO 01/64634, the disclosures each of which are incorporated herein by reference. In a particular embodiment, the CB1 antagonist is rimonabant.

In another embodiment of the present invention, the first pharmaceutical agent is a serotonin receptor agonist, for example a 5HT_{2C}-selective serotonin receptor agonist, that has an anorectic effect. A specific example of a 5HT_{2C}-selective serotonin receptor agonist is lorcaserin (Arena Pharmaceuticals), mesulergine, agomelatine and fluoxetine. Additional 5HT_{2C}-selective serotonin receptor agonists which may be used in the instant invention include BVT933, DPCA37215, 1K264; PNU 22394; WAY161503, R-1065, and YM 348.

### Dosages, Formulations and Administration:

The combination of an anti-epileptic agent, CB1 receptor antagonist, or a 5HT_{2C}-selective serotonin receptor agonist with one or more sympathomimetic agents such as phentermine and/or bupropion provide increased therapeutic effects, and reduced adverse effects, making these pharmaceutical combinations extremely effective therapeutics, especially in the treatment of obesity and related conditions, including conditions associated with and/or caused by obesity per se.

The choice of appropriate dosages for the drugs used in combination therapy according to the present invention can be determined and optimized by the skilled artisan, e.g., by observation of the patient, including the patient's overall health, the response to the combination therapy, and the like. Optimization, for example, may be necessary if it is determined that a patient is not exhibiting the desired therapeutic effect or conversely, if the patient is experiencing undesirable or adverse side effects that are too many in number or are of a troublesome severity.

In one embodiment, each component of the combination (e.g., (i) a sympathomimetic drug, and (ii) an anti-epileptic agent, a CB1 receptor antagonist, or a 5HT_{2C}-selective serotonin receptor agonist) is prescribed at a dose that is below the typically described dose for each component as a monotherapy. The components may be prescribed separately or as a combination dosage. In one embodiment, each component of the combination (e.g., (i) a sympathomimetic drug, and (ii) an anti-epileptic agent, a CB1 receptor antagonist, or a 5HT_{2C}-selective serotonin receptor agonist) is prescribed at a dose that is lower than the typically described dose for each component as a monotherapy. The components may be prescribed separately or as a combination dosage.

In another embodiment, the prescribed dosage of the sympathomimetic drug is above the typically described dose for monotherapy, and the anti-epileptic agent, CB1 receptor antagonist, or 5HT_{2C}-selective serotonin receptor agonist is prescribed at a dosage that is at or below the typically described dose for monotherapy. In another embodiment, the prescribed dosage of the sympathomimetic drug is at or below the typically described dose for monotherapy, and the anti-epileptic agent, CB1 receptor antagonist, or 5HT_{2C}-selective serotonin receptor agonist is prescribed at a dosage that is above the typically described dose for monotherapy.

In certain aspects, the sympathomimetic drug or anti-epileptic agent or CB1 receptor antagonist or 5HT_{2C}-selective serotonin receptor agonist may be administered at a dose ranging from 0.1-500 mg daily, such as from 10-400, and including from 20-400, and including from 50-200, and including from 25-200 mg daily. In other aspects, the sympathomimetic drug or anti-epileptic agent or CB1 receptor antagonist or 5HT_{2C}-selective serotonin receptor agonist may be administered at a dose ranging from 1-250 mg daily, such as from 1-200, and including from 2-100, and including from 2-60, and including from 2-30 mg daily. In another aspect, the sympathomimetic drug or anti-epileptic agent or CB1 receptor atagonist or 5HT_{2C}-selective serotonin receptor agonist may be administered at a dose ranging from 0.1-100 mg daily, such as from 0.2-50 and including from 0.2-25 and including from 0.25-10 and including from 0.25-5 mg daily.

In certain embodiments, when phentermine is the sympathomimetic agent, phentermine may be, for example, administered at a dose ranging from 2-60 mg daily. In one aspect, the phentermine is administered at a dose ranging from 2-30 mg daily.

In certain embodiments, when bupropion is the sympathomimetic agent, bupropion may be, for example, administered at a dose ranging from 50-400 mg daily. In one aspect, the bupropion is administered at a dose ranging from 50-200 mg daily.

In certain embodiments, when topiramate is the first pharmaceutical agent, topiramate may be administered at a dose ranging from 20-400 mg daily. In one aspect, topiramate is administered at a dose ranging from 25-200 mg daily.

In certain embodiments, when zonisamide is the first pharmaceutical agent, zonisamide may be administered at a dose ranging from 20-400 mg daily. In one aspect, zonisamide is administered at a dose ranging from 20-200 mg daily.

In other embodiments, when rimonabant is the first pharmaceutical agent, rimonabant may be administered at dose ranging from 0.25-50 mg daily, such as from 1.0-25 mg daily. In another aspect, rimonabant is administered at dose ranging from 2-15 mg daily, including from 2-10 mg daily, while in other aspects, rimonabant is administered at a dose ranging from 3-7.5 mg daily or from 3-5 mg daily.

In another embodiment, when taranabant is the first pharmaceutical agent, tiranabant may be administered at a dose ranging from 0.25-50 mg daily, such as from 0.25-10 mg daily. In another aspect, taranabant is administered between 0.25-8 mg daily, including from 0.25-7 mg daily, while in other aspects, tiranabant is administered at a dose ranging from 0.25-7.5 mg daily or from 0.25-5 mg daily.

In another embodiment, when lorcaserin is the first pharmaceutical agent, lorcaserin may be administered at a dose ranging from 2-50 mg daily, such as from 2-20 mg daily, including from 5-15 mg daily. In one aspect, lorcaserin is administered at a dose ranging from 2-10 mg daily, including from 3.5-7.5 mg daily.

Further, the patient may receive the specific dosage over a period of weeks, months, or years. For example, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years and the like.

In some embodiments, an “effective amount” of the combination therapy is an amount that results in a reduction of at least one pathological parameter associated with obesity or a related condition. Thus, e.g., in some embodiments, an effective amount of the combination therapy is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter associated with obesity or a related condition.

When administered in separate formulations, the sympathomimetic agent and the anti-epileptic agent, CB1 antagonist, or 5HT_{2C}-selective serotonin receptor agonist may be administered substantially simultaneously (e.g., within about 60 minutes, about 50 minutes, about 40 minutes, about 30 minutes, about 20 minutes, about 10 minutes, about 5 minutes, or about 1 minute of each other) or separated in time by about 1 hour, about 2 hours, about 4 hours, about 6 hours, about 10 hours, about 12 hours, about 24 hours, about 36 hours, or about 72 hours, or more.

It is especially advantageous to formulate compositions of the invention in unit dosage form for ease of administration and uniformity of dosage. The specifications of the novel dosage unit forms of the invention are dependent on the unique characteristics of the composition containing the anti-epileptic, CB1 antagonist, or 5HT_{2C}-selective serotonin receptor agonist and the sympathomimetic agent and the particular therapeutic effect to be achieved. Dosages can further be determined by reference to the usual dose and manner of administration of the ingredients. It is also within the scope of the present invention to formulate a single physically discrete dosage form having each of the active ingredients of the combination treatment (e.g., a single dosage form having an anti-epileptic, CB1 antagonist, or 5HT_{2C}-selective serotonin receptor agonist and the sympathomimetic agent).

The method of administration of compositions or combinations of the invention will depend, in particular, on the type of sympathomimetic agent used and the chosen anti-epileptic agent, CB1 receptor antagonist, or 5HT_{2C}-selective serotonin receptor agonist. The sympathomimetic agent and the anti-epileptic agent, CB1 receptor antagonist, or 5HT_{2C}-selective serotonin receptor agonist may be administered together in the same composition or simultaneously or sequentially in two separate compositions. Also, one or more sympathomimetic agents or one or more anti-epileptic agents, CB1 receptor antagonists, or 5HT_{2C}-selective serotonin receptor agonist may be administered to a subject or patient either in the form of a therapeutic composition or in combination, e.g., in the form of one or more separate compositions administered simultaneously or sequentially. The schedule of administration will be dependent on the type of sympathomimetic agent(s) and anti-epileptic agent(s), CB1 receptor antagonist(s), or 5HT_{2C}-selective serotonin receptor agonist(s) chosen. For example, a sympathomimetic agent can have a stimulant effect and the degree of such stimulant effect may vary depending on the sympathomimetic agent chosen. Accordingly, a sympathomimetic agent having a significant stimulant effect might be administered earlier in the day than administration of a sympathomimetic agent having a lesser stimulant effect. Likewise, an anti-epileptic agent, CB1 receptor antagonist, or 5HT_{2C}-selective serotonin receptor agonist can have a sedative effect and the degree of such sedative effect may vary depending on the compound chosen. Accordingly, an anti-epileptic agent, CB1 receptor antagonist, or 5HT_{2C}-selective serotonin receptor agonist having a significant sedative effect might be administered later in the day than administration of a compound having a lesser sedative effect. Moreover, sympathomimetic agents, anti-epileptic agents, CB1 receptor antagonists, or 5HT_{2C}-selective serotonin receptor agonists having lesser stimulant or sedative effects, respectively, may be administered simultaneously.

In a specific embodiment, topirimate is administered as a controlled release form and phentermine is administered as an immediate release form. As such, the phentermine may be taken in the morning because the drug is a stimulant as well as an appetite suppressant. In this embodiment, topiramate may be taken later in the day than the phentermine. Preferably, the patient takes the topiramate in the afternoon, or just before supper or later in the evening because the drug is sedating.

In another embodiment, zonisamide is administered as a controlled release form and phentermine is administered as an immediate release form. As such, the phentermine may be taken in the morning because the drug is a stimulant as well as an appetite suppressant. In this embodiment, zonisamide may be taken later in the day than the phentermine. Preferably, the patient takes the zonisamide in the afternoon, or just before supper or later in the evening because the drug is sedating.

In another embodiment, rimonabant is administered as a controlled release form and phentermine is administered as an immediate release form. As such, the phentermine may be taken in the morning because the drug is a stimulant as well as an appetite suppressant. In this embodiment, rimonabant may be taken later in the day than the phentermine. Preferably, the patient takes the rimonabant in the afternoon, or just before supper or later in the evening because the drug is sedating.

In another embodiment, lorcaserin is administered as a controlled release form and phentermine is administered as an immediate release form. As such, the phentermine may be taken in the morning because the drug is a stimulant as well as an appetite suppressant. In this embodiment, lorcaserin may be taken later in the day than the phentermine. Preferably, the patient takes the lorcaserin in the afternoon, or just before supper or later in the evening because the drug is sedating.

In yet another embodiment, topirimate is administered as a controlled release form and bupropion is administered as an immediate release form. As such, the bupropion may be taken in the morning because the drug is a stimulant as well as an appetite suppressant. In this embodiment, topiramate may be taken later in the day than the bupropion. Preferably, the patient takes the topiramate in the afternoon, or just before supper or later in the evening because the drug is sedating.

In another embodiment, zonisamide is administered as a controlled release form and bupropion is administered as an immediate release form. As such, the bupropion may be taken in the morning because the drug is a stimulant as well as an appetite suppressant. In this embodiment, zonisamide may be taken later in the day than the bupropion. Preferably, the patient takes the zonisamide in the afternoon, or just before supper or later in the evening because the drug is sedating.

In another embodiment, rimonabant is administered as a controlled release form and bupropion is administered as an immediate release form. As such, the bupropion may be taken in the morning because the drug is a stimulant as well as an appetite suppressant. In this embodiment, rimonabant may be taken later in the day than the bupropion. Preferably, the patient takes the rimonabant in the afternoon, or just before supper or later in the evening because the drug is sedating.

In another embodiment, lorcaserin is administered as a controlled release form and bupropion is administered as an immediate release form. As such, the bupropion may be taken in the morning because the drug is a stimulant as well as an appetite suppressant. In this embodiment, lorcaserin may be taken later in the day than the bupropion. Preferably, the patient takes the lorcaserin in the afternoon, or just before supper or later in the evening because the drug is sedating.

Administration of the active agent may be carried out using any appropriate mode of administration. Thus, administration can be, for example, oral, parenteral, transdermal, transmucosal (including rectal, vaginal, and transurethral), sublingual, by inhalation, or via an implanted reservoir in a dosage form. The term “parenteral” as used herein is intended to include subcutaneous, intravenous, and intramuscular injection.

Depending on the intended mode of administration, the pharmaceutical formulation may be a solid, semi-solid or liquid, such as, for example, a tablet, a capsule, a caplet, a liquid, a suspension, an emulsion, a suppository, granules, pellets, beads, a powder, or the like, preferably in unit dosage form suitable for single administration of a precise dosage. Suitable pharmaceutical compositions and dosage forms may be prepared using conventional methods known to those in the field of pharmaceutical formulation and described in the pertinent texts and literature, e.g., in Remington: The Science and Practice of Pharmacy (Easton, Pa.: Mack Publishing Co., 1995). For those compounds that are orally active, oral dosage forms are generally preferred, and include tablets, capsules, caplets, solutions, suspensions and syrups, and may also comprise a plurality of granules, beads, powders, or pellets that may or may not be encapsulated. Preferred oral dosage forms are tablets and capsules.

As noted above, it is especially advantageous to formulate compositions of the invention in unit dosage form for ease of administration and uniformity of dosage. The term “unit dosage forms” as used herein refers to physically discrete units suited as unitary dosages for the individuals to be treated. That is, the compositions are formulated into discrete dosage units each containing a predetermined, “unit dosage” quantity of an active agent calculated to produce the desired therapeutic effect in association with the required pharmaceutical carrier. The specifications of unit dosage forms of the invention are dependent on the unique characteristics of the active agent to be delivered. Dosages can further be determined by reference to the usual dose and manner of administration of the ingredients. It should be noted that, in some cases, two or more individual dosage units in combination provide a therapeutically effective amount of the active agent, e.g., two tablets or capsules taken together may provide a therapeutically effective dosage of the first pharmaceutical active agent, e.g., anti-epileptic agent, CB1 receptor antagonist, or a 5HT_{2C}-selective serotonin receptor agonist or the second pharmaceutical agent, e.g., the sympathomimetic agent, e.g., phentermine or bupropion, such that the unit dosage in each tablet or capsule is approximately 50% of the therapeutically effective amount.

Tablets may be manufactured using standard tablet processing procedures and equipment. Direct compression and granulation techniques are preferred. In addition to the active agent, tablets will generally contain inactive, pharmaceutically acceptable carrier materials such as binders, lubricants, disintegrants, fillers, stabilizers, surfactants, coloring agents, and the like.

Capsules are also preferred oral dosage forms for those pharmaceutical active agents that are orally active, in which case the active agent-containing composition may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited earlier herein, which describes materials and methods for preparing encapsulated pharmaceuticals.

Oral dosage forms, whether tablets, capsules, caplets, or particulates, may, if desired, be formulated so as to provide for controlled release of the first pharmaceutical active agent, e.g., anti-epileptic agent, CB1 receptor antagonist, or a 5HT_{2C}-selective serotonin receptor agonist or the second pharmaceutical agent, e.g., the sympathomimetic agent, e.g., phentermine or bupropion, and in a preferred embodiment, the present formulations are controlled release oral dosage forms. Generally, the dosage forms provide for sustained release, i.e., gradual, release of the first pharmaceutical active agent, e.g., anti-epileptic agent, CB1 receptor antagonist, or a 5HT_{2C}-selective serotonin receptor agonist or the second pharmaceutical agent, e.g., the sympathomimetic agent, e.g., phentermine or bupropion, from the dosage form to the patient's body over an extended time period, typically providing for a substantially constant blood level of the agent over a time period in the range of about 4 to about 12 hours, typically in the range of about 6 to about 10 hours. In a particularly preferred embodiment, there is a very gradual increase in blood level of the drug following oral administration of the dosage form containing the first pharmaceutical active agent, e.g., anti-epileptic agent, CB1 receptor antagonist, or a 5HT_{2C}-selective serotonin receptor agonist or the second pharmaceutical agent, e.g., the sympathomimetic agent, e.g., phentermine or bupropion, such that peak blood level is not reached until at least 4-6 hours have elapsed, with the rate of increase of blood level drug approximately linear, e.g., (generally about 50-200 μg/ml for topiramate, about 1-5 μg/ml for zonisamide, or about 10-35 μg/ml for acetazolamide). In addition, in the preferred embodiment, there is an equally gradual decrease in blood level at the end of the sustained release period.

Generally, as will be appreciated by those of ordinary skill in the art, sustained release dosage forms are formulated by dispersing the active agent within a matrix of a gradually hydrolyzable material such as a hydrophilic polymer, or by coating a solid, drug-containing dosage form with such a material. Hydrophilic polymers useful for providing a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

Preferred sustained release dosage forms herein are composed of the acrylate and methacrylate copolymers available under the tradename “Eudragit” from Rohm Pharma (Germany). The Eudragit series E, L, S, RL, RS, and NE copolymers are available as solubilized in organic solvent, in an aqueous dispersion, or as a dry powder. Preferred acrylate polymers are copolymers of methacrylic acid and methyl methacrylate, such as the Eudragit L and Eudragit S series polymers. Particularly preferred such copolymers are Eudragit L-30D-55 and Eudragit L-100-55 (the latter copolymer is a spray-dried form of Eudragit L-30D-55 that can be reconstituted with water). The molecular weight of the Eudragit L-30D-55 and Eudragit L-100-55 copolymer is approximately 135,000 Da, with a ratio of free carboxyl groups to ester groups of approximately 1:1. The copolymer is generally insoluble in aqueous fluids having a pH below 5.5. Another particularly suitable methacrylic acid-methyl methacrylate copolymer is Eudragit S-100, which differs from Eudragit L-30D-55 in that the ratio of free carboxyl groups to ester groups is approximately 1:2. Eudragit S— 100 is insoluble at pH below 5.5, but unlike Eudragit L-30D-55, is poorly soluble in aqueous fluids having a pH in the range of 5.5 to 7.0. This copolymer is soluble at pH 7.0 and above. Eudragit L-100 may also be used, which has a pH-dependent solubility profile between that of Eudragit L-30D-55 and Eudragit S-100, insofar as it is insoluble at a pH below 6.0. It will be appreciated by those skilled in the art that Eudragit L-30D-55, L-100-55, L-100, and S-100 can be replaced with other acceptable polymers having similar pH-dependent solubility characteristics. Other preferred Eudragit polymers are cationic, such as the Eudragit E, RS, and RL series polymers. Eudragit E100 and E PO are cationic copolymers of dimethylaminoethyl methacrylate and neutral methacrylates (e.g., methyl methacrylate), while Eudragit RS and Eudragit RL polymers are analogous polymers, composed of neutral methacrylic acid esters and a small proportion of trimethylammonioethyl methacrylate.

Preparations according to this invention for parenteral administration include sterile aqueous and nonaqueous solutions, suspensions, and emulsions. Injectable aqueous solutions contain the active agent in water-soluble form. Examples of nonaqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations are rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

The active agent may also be administered through the skin using conventional transdermal drug delivery systems, wherein the active agent is contained within a laminated structure that serves as a drug delivery device to be affixed to the skin. In such a structure, the drug composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. Transdermal drug delivery systems may in addition contain a skin permeation enhancer.

In addition to the formulations described previously, the active agent may be formulated as a depot preparation for controlled release of the active agent, preferably sustained release over an extended time period. These sustained release dosage forms are generally administered by implantation (e.g., subcutaneously or intramuscularly or by intramuscular injection).

Although the present compositions will generally be administered orally, parenterally, transdermally, or via an implanted depot, other modes of administration are suitable as well. For example, administration may be transmucosal, e.g., rectal or vaginal, preferably using a suppository that contains, in addition to the active agent, excipients such as a suppository wax. Transmucosal administration also encompasses transurethral administration, as described, for example, in U.S. Pat. Nos. 5,242,391, 5,474,535, and 5,773,020 to Place et al. Formulations for nasal or sublingual administration are also prepared with standard excipients well known in the art. The pharmaceutical compositions of the invention may also be formulated for inhalation, e.g., as a solution in saline, as a dry powder, or as an aerosol.

### Indications:

While the invention is useful in conjunction with numerous pharmaceutical agents and therapeutic regimens, conditions of particular interest include obesity and related conditions, such as those often associated with and/or caused by obesity. The combination of an anti-epileptic agent, CB1 receptor antagonist, or a 5HT_{2C}-selective serotonin receptor agonist with one or more sympathomimetic agents such as phentermine and/or bupropion provide increased therapeutic effects, and reduced adverse effects, making these pharmaceutical combinations extremely effective therapeutics, especially in the treatment of obesity and related conditions, including conditions associated with and/or caused by obesity per se.

As such, combination treatment may be employed to decrease the doses of the individual components in the resulting combinations while still preventing unwanted or harmful side effects of the individual components. Moreover, combination treatment offers a choice of various drugs for treating obesity or a related condition. As such, when one drug combination does not work in a particular individual, another combination may be administered which will be effective for treating obesity or a related condition. Accordingly, the drug combinations of the present invention provides a choice of various drugs for an individual who does not react to one particular combination or experiences adverse side effects. As such, a second combination of drugs may be administered to an individual when a first drug combination was not effective for treating obesity or a related condition.

Subjects suitable for treatment with the subject combination therapy treatment regimen include individuals suffering from the following conditions associated with obesity, including, hypertension, diabetes or glucose intolerance and insulin resistance, hyperlipidemia, and often tiredness and sleepiness associated with sleep apnea. Patients are often treated with combinations of antihypertensives, lipid lowering agents, insulin or oral diabetic drugs, and various mechanical and surgical methods for treating sleep apnea. However, such treatments are often costly and do not treat the underlying problem of obesity. Moreover, some of the treatments for diabetes including insulin and oral diabetic agents actually aggravate the conditions associated with obesity by increasing insulin levels, increasing appetite, and increasing weight. This can lead to higher blood pressure and even higher cholesterol.

### Overweight and Obesity:

“Overweight and Obesity” are defined by the Centers for Disease Control and Prevention (the CDC) with respect to body mass index, or BMI, of a patient. Factors such as weight and height are used to determine whether a person is underweight, normal, overweight or obese. The CDC also notes other methods for analysis of amount and location of fat in a person by using measurements of skinfold thickness and waist circumference, waist-to-hip circumference ratios, and techniques such as ultrasound, computed tomography, and magnetic resonance imaging (MRI). Much money has been invested by both companies providing weight loss programs and products, and patients attempting to lose weight. Almost all aspects of a person are affected by overweight and obesity, from physical problems such as knee and ankle joint deterioration, to emotional problems due to society's rejection of overweight

Symptoms of overweight and obesity are usually quite obvious, as excess fat is often easy to see on a person individuals. The medical problems caused by overweight and obesity can be serious and often life-threatening, and include diabetes, shortness of breath and other respiratory problems, gallbladder disease, hypertension, dyslipidemia (for example, high cholesterol or high levels of triglycerides), cancer, osteoarthritis, other orthopedic problems, reflux esophagitis (heartburn), snoring, sleep apnea, menstrual irregularities, infertility and heart trouble. Moreover, obesity and overweight substantially increase the risk of morbidity from hypertension, dyslipidemia, type 2 diabetes, coronary heart disease, stroke, gallbladder disease, osteoarthritis and endometrial, breast, prostate, and colon cancers. Higher body weights are also associated with increases in all-cause mortality. Most or all of these problems are relieved or improved by permanent significant weight loss. Longevity is likewise significantly increased by permanent significant weight loss.

### Diabetes:

Diabetes mellitus is associated with continuous and pathologically elevated blood glucose concentration. It is one of the leading causes of death in the United States and is responsible for about 5% of all mortality. Diabetes is divided into two major sub-classes: Type I, also known as juvenile diabetes, or Insulin-Dependent Diabetes Mellitus (IDDM); and Type II, also known as adult onset diabetes, or Non-Insulin-Dependent Diabetes Mellitus (NIDDM).

According to the American Diabetes Association, there are over one million juvenile diabetics in the United States. Type I Diabetes is a form of autoimmune disease. Autoantibodies produced by the patients completely or partially destroy the insulin producing cells of the pancreas. Juvenile diabetics must, therefore, receive exogenous insulin during their lifetime. Without treatment, excessive acidosis, dehydration, kidney damage, and death may result. Even with treatment, complications such as blindness, atherosclerosis, and impotence can occur.

There are more than five million Type II (adult onset) diabetics diagnosed in the United States. Type II disease usually begins during middle age; the exact cause is unknown. In Type II diabetics, rising blood glucose levels after meals do not properly stimulate insulin production by the pancreas. Additionally, peripheral tissues are generally resistant to the effects of insulin. The resulting high blood glucose levels (hyperglycemia) can cause extensive tissue damage. Type II diabetics are often referred to as insulin resistant. They often have higher than normal plasma insulin levels (hyperinsulinemia) as the body attempts to overcome its insulin resistance. Some researchers now believe that hyperinsulinemia may be a causative factor in the development of high blood pressure, high levels of circulating low density lipo-proteins (LDLs), and lower than normal levels of the beneficial high density lipo-proteins (HDLs). While moderate insulin resistance can be compensated for in the early stages of Type II diabetes by increased insulin secretion, in advanced disease states insulin secretion is also impaired.

Insulin resistance and hyperinsulinemia have also been linked with two other metabolic disorders that pose considerable health risks: impaired glucose tolerance and metabolic obesity. Impaired glucose tolerance is characterized by normal glucose levels before eating, with a tendency toward elevated levels (hyperglycemia) following a meal. According to the World Health Organization, approximately 11% of the U.S. population between the ages of 20 and 74 are estimated to have impaired glucose tolerance. These individuals are considered to be at higher risk for diabetes and coronary artery disease. In certain aspects, the subject invention may be employed for treating pre-diabetes such that administering the invention to a patient may prevent the onset of diabetes.

Obesity may also be associated with insulin resistance. A causal linkage among obesity, impaired glucose tolerance, and Type II diabetes has been proposed, but a physiological basis has not yet been established. Some researchers believe that impaired glucose tolerance and diabetes are clinically observed and diagnosed only later in the disease process after a person has developed insulin resistance and hyperinsulinemia.

Insulin resistance is frequently associated with hypertension, coronary artery disease (arteriosclerosis), and lactic acidosis, as well as related disease states. The fundamental relationship between these disease states, and a method of treatment, has not been established.

### Diabetic Nephropathy:

Diabetic nephropathy (nephropatia diabetica), also known as Kimmelstiel-Wilson syndrome and intercapillary glomerulonephritis, is a progressive kidney disease caused by angiopathy of capillaries in the kidney glomeruli. It is characterized by nephrotic syndrome and nodular glomerulosclerosis. It is due to longstanding diabetes mellitus, and is a prime cause for dialysis in many Western countries

### Hypertension:

Hypertension is a condition that occurs when the blood pressure inside the large arteries is too high. Hypertension is very common, affecting about 50 million people in the United States alone. It is more common as people grow older and is both more common and more serious in African Americans. Most cases of hypertension are of unknown etiology. It is known that the tendency to develop hypertension can be inherited. Environment also plays a very important role in hypertension. For example, hypertension may be avoided by keeping body weight under control, keeping physically fit, eating a healthy diet, limiting alcohol intake, and avoiding medications that might increase blood pressure. Other less common causes of hypertension include disorders of the kidneys or endocrine glands. Hypertension has been called “the silent killer” because it has no specific symptoms and yet can lead to death. People with untreated hypertension are much more likely to die from or be disabled by cardiovascular complications such as strokes, heart attacks, heart failure, heart rhythm irregularities, and kidney failure, than people who have normal blood pressure.

Current treatments for hypertension include lifestyle changes (diet, exercise, nonsmoking, etc.) as well as drug therapy. The major classes of medications currently used to treat hypertension include adrenergic neuron antagonists (which are peripherally acting), alpha adrenergic agonists (which are centrally acting), alpha adrenergic blockers, alpha and beta blockers, angiotensin II receptor blockers, angiotensin converting enzyme (ACE) inhibitors, beta adrenergic blockers, calcium channel blockers, thiazides (benzothiadiazine derivatives) and related diuretics, and vasodilators (which act by direct relaxation of vascular smooth muscles).

A particularly serious hypertensive disorder is primary pulmonary hypertension, also known as idiopathic pulmonary hypertension. This is a condition in which the blood pressure in the pulmonary arteries is abnormally high in the absence of other diseases of the heart or lungs. The cause of primary pulmonary hypertension is unknown. Pulmonary hypertension develops in response to increased resistance to blood flow. Narrowing of the pulmonary arterioles occurs and the right side of the heart becomes enlarged due to the increased work of pumping blood against the resistance. Eventually, progressive heart failure develops. Currently, there is no known cure for primary pulmonary hypertension. Treatment is primarily directed towards controlling the symptoms, although some success has occurred with the use of vasodilators. Other medications used to treat the symptoms of primary pulmonary hypertension include diuretics and calcium channel blockers. Typically, as the disease progresses, oxygen is often required. In certain cases, a heart-lung transplant may be indicated for certain suitable candidates, although the availability of donor organs continues to be extremely limited. Unfortunately, primary pulmonary hypertension is a progressive disease, usually leading to congestive heart failure and respiratory failure.

Secondary pulmonary hypertension is a serious disorder that arises as a complication of other conditions such as, for example, scleroderma. Treatments are similar as those for primary pulmonary hypertension and, unfortunately, the prognosis is the same as well.

### Sleep Apnea:

Sleep apnea occurs in two main types: obstructive sleep apnea, the more common form that occurs when throat muscles relax; and central sleep apnea, which occurs when the brain doesn't send proper signals to the muscles that control breathing. Additionally, some people have mixed sleep apnea, which is a combination of both obstructive and central sleep apneas. Sleep apnea literally means “cessation of breath.” It is characterized by repetitive episodes of upper airway obstruction that occur during sleep, usually associated with a reduction in blood oxygen saturation. In other words, the airway becomes obstructed at several possible sites. The upper airway can be obstructed by excess tissue in the airway, large tonsils, a large tongue and usually includes the airway muscles relaxing and collapsing when asleep. Another site of obstruction can be the nasal passages. Sometimes the structure of the jaw and airway can be a factor in sleep apnea.

The signs and symptoms of obstructive and central sleep apneas overlap, sometimes making the type of sleep apnea more difficult to determine. The most common signs and symptoms of obstructive and central sleep apneas include: excessive daytime sleepiness (hypersomnia); loud snoring; observed episodes of breathing cessation during sleep; abrupt awakenings accompanied by shortness of breath; awakening with a dry mouth or sore throat; morning headache; and/or difficulty staying asleep (insomnia). Disruptive snoring may be a more prominent characteristic of obstructive sleep apnea, while awakening with shortness of breath may be more common with central sleep apnea.

Sleep apnea is a progressive condition and should not be taken lightly. It is a potentially life-threatening condition that requires immediate medical attention. The risks of undiagnosed obstructive sleep apnea include heart attacks, strokes, impotence, irregular heartbeat, high blood pressure and heart disease. In addition, obstructive sleep apnea causes daytime sleepiness that can result in accidents, lost productivity and interpersonal relationship problems. The severity of the symptoms may be mild, moderate or severe.

Sleep apnea is diagnosed utilizing a sleep test, called polysomnography but treatment methodologies differ depending on the severity of the disorder. Mild Sleep Apnea is usually treated by some behavioral changes. Losing weight, sleeping on your side are often recommended. There are oral mouth devices (that help keep the airway open) that may help to reduce snoring in three different ways. Some devices (1) bring the jaw forward or (2) elevate the soft palate or (3) retain the tongue (from falling back in the airway and blocking breathing).

Moderate to severe sleep apnea is usually treated with a continuous positive airway pressure (C-PAP). C-PAP is a machine that blows air into your nose via a nose mask, keeping the airway open and unobstructed. For more severe apnea, there is a Bi-level (Bi-PAP) machine. The Bi-level machine is different in that it blows air at two different pressures. When a person inhales, the pressure is higher and in exhaling, the pressure is lower. Some people have facial deformities that may cause the sleep apnea. It simply may be that their jaw is smaller than it should be or they could have a smaller opening at the back of the throat. Some people have enlarged tonsils, a large tongue or some other tissues partially blocking the airway. Fixing a deviated septum may help to open the nasal passages. Removing the tonsils and adenoids or polyps may help also. Children are much more likely to have their tonsils and adenoids removed. Surgical procedures, such as tracheostomy, uvulopalatopharyngoplasty (UPPP), laser assisted uvuloplasty (LAUP), somnoplasty, or mandibular myotomy, are often required to effectively treat sleep apnea.

### Epilepsy:

“Epilepsy” is defined as a brain disorder with recurrent, unprovoked seizures. Epilepsy includes seizures of focal onset and generalized seizures. The types of focal onset seizures are partial seizures of temporal lobe origin, frontal lobe origin or others. Focal epilepsies with genetic components include benign childhood epilepsy with centrotemporal spikes, childhood epilepsy with occipital paroxysms or primary reading epilepsy. The generalized genetic epilepsies include benign neonatal familial convulsions, benign neonatal convulsions, benign myoclonic epilepsy in infancy, childhood absence epilepsy, juvenile absence epilepsy, juvenile myoclonic epilepsy, and epilepsy with grand mal seizures on awakening. The cryptogenic childhood epilepsies with generalized seizures include infantile spasms without tuberous sclerosis, West syndrome, Lennox-Gastaut syndrome, epilepsy with myoclonic-astatic seizures, epilepsy with myoclonic absences, and symptomatic epilepsy such as infantile spasms associated with tuberous sclerosis, epilepsy with continuous spike and wave EEG during slow-wave sleep, and acquired epileptic aphasia (Landau-Kleffner syndrome).

The treatment of any convulsive disorders is also contemplated using the methods and compositions of the invention. These convulsive disorders include all forms of epilepsies, for example, temporal lobe epilepsy, focal epilepsies, including idiopathic epilepsies such as benign childhood epilepsy with centrotemporal spikes, childhood epilepsy with occipital paroxysms or primary reading epilepsy, symptomatic epilepsies with simple partial seizures, complex partial seizures, secondarily generalized seizures, generalized epilepsies and syndromes; generalized epilepsies including idiopathic epilepsies such as benign neonatal familial convulsions, benign neonatal convulsions, benign myoclonic epilepsy in infancy, childhood absence epilepsy, juvenile absence epilepsy, juvenile myoclonic epilepsy, and epilepsy with grand mal seizures on awakening; cryptogenic or symptomatic epilepsies including West syndrome, Lennox-Gastaut syndrome, epilepsy with myoclonic-astatic seizures, epilepsy with myoclonic absences, and symptomatic epilepsy such as early myoclonic encephalopathy or specific syndromes; epilepsies with undetermined origin including neonatal seizures, severe myoclonic epilepsy in infancy, epilepsy with continuous spike and wave EEG during slow-wave sleep, and acquired epileptic aphasia (Landau-Kleffner syndrome). It also includes seizures caused by metabolic derangements, acute brain injury, acute head trauma, drug withdrawal, alcohol withdrawal, and toxins.

Symptoms associated with, or arising from epilepsy, include convulsions, grand mal seizures, absence seizures, petit mal seizures, focal seizures, temporal lobe seizures, psychomotor seizures, muscle spasms, loss of consciousness, strange sensations, strange emotions and strange behavior.

### Migraines:

“Migraines” are generally headaches that are typically associated with various psychological (e.g., irritability, depression, fatigue, drowsiness, and restlessness), neurological (e.g., photophobia, and phonophobia), and gastrointestinal symptoms. The headache starts with mild pain, which increases in intensity over a short period of time. There are two major types of migraines. The common migraine affects 80-85% of migraine sufferers and classical migraine with aura affects 15% of migraine sufferers. Symptoms associated with migraines include headaches, psychological symptomatology such as irritability, depression, fatigue, drowsiness, restlessness; neurological symptoms such as photophobia, phonophobia or gastrointestinal symptoms such as change in bowel habit, change of food intake or urinary symptoms such as urinary frequency, auras which are neurological deficits and can be a variety of deficits for the migraine population but in the individual is usually stereotyped. These deficits may be visual scotoma or visual designs, hemiplegia, migrating paraesthesia, dysarthria, dysphasia, or deja-vu. The headache is usually accompanied by light or sound sensitivity, photophobia or phonophobia, irritability and impaired concentration.

### Depression:

“Depression” is manifested by a combination of symptoms that interfere with the ability to work, study, sleep, eat, and enjoy once pleasurable activities. Depression includes major depression, especially refractory depression, bipolar depression, and the degeneration associated with depression. Symptoms of depression include persistent sad, anxious, or “empty” mood, feelings of hopelessness, pessimism, feelings of guilt, worthlessness, helplessness, loss of interest or pleasure in hobbies and activities that were once enjoyed, including sex, decreased energy, fatigue, being “slowed down”, difficulty concentrating, remembering, making decisions, insonmia, early-morning awakening, or oversleeping, appetite and/or weight loss or overeating and weight gain, thoughts of death or suicide; suicide attempts, restlessness, irritability, persistent physical symptoms that do not respond to treatment, such as headaches, digestive disorders, and chronic pain.

### Nonalcoholic Steatohepatitis and Nonalcoholic Fatty Liver Disease:

Nonalcoholic steatohepatitis (NASH) is a common, often “silent” liver disease. It resembles alcoholic liver disease, but occurs in people who drink little or no alcohol. The major feature in NASH is fat in the liver, along with inflammation and damage. Most people with NASH feel well and are not aware that they have a liver problem. Nevertheless, NASH can be severe and can lead to cirrhosis, in which the liver is permanently damaged and scarred and no longer able to work properly.

NASH affects 2 to 5 percent of Americans. An additional 10 to 20 percent of Americans have fat in their liver, but no inflammation or liver damage, a condition called “fatty liver.” Although having fat in the liver is not normal, by itself it probably causes little harm or permanent damage. If fat is suspected based on blood test results or scans of the liver, this problem is called nonalcoholic fatty liver disease (NAFLD). If a liver biopsy is performed in this case, it will show that some people have NASH while others have simple fatty liver.

### Others.

Other psychiatric disorders may also be treated using the compositions and methods of the invention. These disorders include panic syndrome, general anxiety disorder, phobic syndromes of all types, mania, manic depressive illness, hypomania, unipolar depression, stress disorders, PTSD, somatoform disorders, personality disorders, psychosis, and schizophrenia.

“Impulse Control Disorders” are characterized by harmful behaviors performed in response to irresistible impulses. The essential feature of an impulse control disorder is the failure to resist an impulse, drive, or temptation to perform an act that is harmful to the person or to others. Symptoms include an increasing sense of tension or arousal before committing an act, and then experiences pleasure, gratification, or release at the time of committing the act. After the act is performed, there may or may not be regret or guilt. Numerous disorders can be characterized as impulse control disorders including intermittent explosive disorder, kleptomania, pathological gambling, pyromania, trichotillomania, compulsive buying or shopping, repetitive self-mutilation, nonparaphilic sexual addictions, severe nail biting, compulsive skin picking, personality disorders with impulsive features, attention deficit/hyperactivity disorder, eating disorders characterized by binge eating, and substance use disorders.

“Alcohol addiction” is characterized in a subject by the presence of one or more of the following symptoms. The subject has a tolerance for alcohol. The subject has withdrawal symptoms after stopping drinking alcohol. The subject takes alcohol in larger amounts than was intended. The subject lacks the ability to decrease the amount of alcohol consumed. The subject spends a great deal of time attempting to acquire alcohol. Lastly, the subject continues to use alcohol even though the subject should know that there are reoccurring physical or psychological problems being caused by the alcohol.

Symptoms associated with alcohol addiction include death from alcohol toxemia, cirrhosis of the liver, pancreatitis, heart disease, polyneuropathy, alcoholic dementia, increased incidence of many types of cancer: breast cancer, head and neck cancer, esophageal cancer and colorectal cancer, nutritional deficiency involving deficiencies in folic acid, thiamine (vitamin B1), sexual dysfunction, osteoporosis and osteonecrosis.

### Kits:

Also provided are kits for practicing the subject methods. The subject kits may vary greatly in regards to the components included. The subject kits at least include a first pharmaceutical agent and a second pharmaceutical agent, wherein the second pharmaceutical agent is a sympathomimetic agent and wherein the first pharmaceutical agent is an anti-epileptic agent, CB1 receptor antagonist, or a 5HT_{2C}-selective serotonin receptor agonist. In certain embodiments, the subject kits include instructions for a patient to carry out drug administration to treat obesity and/or those conditions associated with obesity. The instructions may be recorded on a suitable recording medium or substrate. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate

Some or all components of the subject kits may be packaged in suitable packaging to maintain sterility. In many embodiments of the subject kits, the components of the kit are packaged in a kit containment element to make a single, easily handled unit, where the kit containment element, e.g., box or analogous structure, may or may not be an airtight container, e.g., to further preserve the sterility of some or all of the components of the kit. In certain aspects, the subject kit comprises a sealed package of controlled release dosage forms wherein the dosage forms provide for immediate release of the second pharmaceutical agent and delayed release of the first pharmaceutical agent.

This invention is further illustrated by the following examples which should not be construed as limiting. The contents of all references, patents and published patent applications cited throughout this application are hereby incorporated by reference.

### EXAMPLE 1

A combination of Topiramate and Bupropion was administered to a patient seeking to lose weight. Subject 1: A male patient suffering from obesity had a starting body weight of 245 lbs., an initial Body Mass Index (BMI) of 36 and a Baseline Blood Pressure of (BP) 122/60. Patient sought treatment for weight loss. Patient was administered a combination of Bupropion and Topiramate according to the following dosing regimen:
150 mg of Bupropion (slow release) and 25 mg of Topiramate (administered at night) for week 1;
150 mg of Bupropion (2 times a day) and gradual increase of Topiramate to a 100 mg (administered at night) over the next four weeks and then maintain dosage for a specific period of time (approximately 10 weeks);
150 mg of Bupropion (2 times a day) and 150 mg of Topiramate for a specific period of time.

A first follow-up visit occurred approximately 4.5 weeks after starting treatment. Patient weighed 228 lbs. and had a BP of 116/60. In a second follow-up visit, patient weighed 230 lbs and had a BP of 102/70.

The combination of Topiramate and Bupropion has enabled the patient to lose weight consistently. As such, the therapeutic efficacy of the combination of Topiramate and Bupropion is improved over the administration of either drug alone.

### EXAMPLE 2

A patient seeking to lose weight was initially administered a combination of Phentermine and Topiramate. Subject 2: A male patient suffering from obesity had a starting body weight of 226 lbs., an initial BMI of 29 and a BP of 122/90. This patient had a history of hypertension and was currently taking 20 mg of Lotensin daily. Patient was administered the typical starting doses of Phentermine and Topiramate ending with a dose of 15 mg of Phentermine in the morning and 100 mg of Topiramate at night. During a follow-up visit, patient stated that he was unable to maintain the treatment continuously because he was suffering from various side effects, such as erection dysfunction (ED), insomnia, and anxiety.

In response, patient discontinued taking Phentermine and instead received a daily dose of 300 mg of Bupropion XL (extended-release) in the morning while continuing with the 100 mg of Topiramate at night.

In follow up visits over the next several months, patient had less problems with anxiety, ED, and anxiety while maintaining a weight of 204 lbs and a BP of 125/70.

Although patient had initial problems with Phentermine, the combination of Topiramate and Bupropion has enabled the patient to lose weight consistently with a reduction in adverse side effects over the administration of either drug alone.

### EXAMPLE 3

A combination of Topiramate and Bupropion was administered to a patient seeking to lose weight. Subject 3: A female patient suffering from obesity had a starting body weight of 284 lbs., an initial BMI of 45½ and a BP of 122/76. This patient also suffered from hypertension, sleep apnea, depression and pulmonary hypertension. Patient was receiving the following baseline medications: 80 mg/day of Lasix, 10 mg/day of Lisinopril, 10 mg/day of Lexapro, 25 mg of Coreg (2×/day), and 0.5 mg of Xanax (3×/day). Patient was administered a combination of Bupropion and Topiramate according to the following dosing regimen:
150 mg of Bupropion (extended release) in the morning and 25 mg of Topiramate (administered in the afternoon) and gradually increasing to a 100 mg over the next four weeks and then maintained for a specific period of time;
about half-way through the treatment, Phentermine was added at 5 mg/day. Patient initially started taking 81 mg/day of ASA and instructed to gradually decrease her Lasix as her BP would decrease as she was placed on the meds and started to lose weight.

Over the next year, patient reduced her pulmonary pressure from greater than 70 mm Hg to normal. Patient experienced a better tolerance for exercise and no longer required a CPAP machine. Approximately, 13 months after starting treatment, patient weighed 212 lbs. and had a BP of 120/80. Patient was further able to reduce her BP meds.

The combination of Topiramate and Bupropion, with the addition of Phentermine half way through the treatment, has enabled the patient to lose weight while also treating various conditions and disorders related to obesity. In addition, the therapeutic efficacy of the combination of Topiramate, Bupropion and Phentermine is improved over the administration of either of the drugs alone.

### EXAMPLE 4

A patient seeking to lose weight was initially administered a combination of Topiramate and Bupropion. Subject 4: A female patient suffering from obesity had a starting body weight of 222 lbs. and an initial BMI of 33. This patient had a history of obesity, hypertension, elevated lipids and depression. Patient was administered the typical starting does of Phentermine and Topiramate. Patient was also taking the following baseline meds: 50 mg/day of Cozaar, 20 mg of Prozac (2×/day). Over a period of 3 years, patient was unable to maintain the treatment continuously but did reach a weight of 207 lbs.

In a follow up visit, patient started taking Zonisamide, discontinued taking Topiramate and continued to take the Phentermine as before. During another follow up visit, patient weighed 201 lbs., felt less depressed and had a decreased appetite for carbohydrates and sweets.

Although patient had initial problems with Phentermine and Topiramate, the combination of Zonisamide and Phentermine has enabled the patient to lose weight while also treating various conditions and disorders related to obesity. In addition, the therapeutic efficacy of the combination of Zonisamide and Phentermine is improved over the administration of either drug alone.

### EXAMPLE 5

A patient seeking to lose weight was initially administered a combination of Phentermine and Topiramate. Subject 5: A female patient suffering from obesity had a starting body weight of 185 lbs., an initial BMI of 33 and a BP of 140/72. This patient also suffered from hypertension and chronic lymphocytic leukemia (CLL). Patient was receiving the following baseline medications: 25 mg/day of HCTZ, 10 mg/day of Prempro, and 10 mg/day of Norvasc. Patient was administered an initial therapy of a combination of Phentermine (½ of a 37.5 mg tab) and Topiramate, which was dose-escalated to 100 mg/day. For approximately 10 months, patient demonstrated steady and regular improvement in weight, BP, and improvements in CLL, such that patient weighed 148 lbs. and had a BP of 110/70. Patient was no longer taking Norvasc and HCTZ.

Towards the end of 1 year, patient developed a rash from Topiramate. As such, patient discontinued taking Topiramate. During a follow up visit just after discontinuing Topiramate, patient felt slightly more hungry, weighed 148 lbs., and had a BP of 140/82. At this time, patient was only taking Phentermine alone.

In response, patient started taking 100 mg/day of Zonisamide at bedtime. During another follow up visit, patient stated that her appetite was better controlled, her weight was 146 lbs., and her BP was 140/60. Further, patient was no longer taking any of her BP medications.

During the course of treatment, patient had the following White Blood Cell Counts and lymphocyte percentages:

| | WHITE BLOOD | |
|---|---|---|
| DATE | CELL COUNT | % LYMPHOCYTES |
| Apr. 18, 2002 (just prior to | 14,000 | 66 |
| initial start) | | |
| Sep. 10, 2002 | 12,200 | 62 |
| Aug. 25, 2003 | 9,000 | 45 |
| Dec. 2, 2003 | 8,000 | 40 |
| Oct. 26, 2004 (discontinued | 11,300 | 53 |
| Topiramate for over 1 year) | | |

As noted above, on Oct. 26, 2004, patient had increased WBC to 11,300 with 53% lymphs. This strongly suggests that the Topiramate played a role in controlling patient's CLL while she was taking it.

Although patient had initial problems with Phentermine and Topiramate, the combination of Zonisamide and Phentermine has enabled the patient to lose weight while also treating various conditions and disorders related to obesity. In addition, the therapeutic efficacy of the combination of Zonisamide and Phentermine is improved over the administration of either drug alone.

### EXAMPLE 6

The subject combination therapy may be administered, unless otherwise indicated, by conventional therapeutic regimens and the like, which are within the skill of the art. Such techniques are fully explained in the literature. See, for example, JAAM. 2006; 295:761-775.

The effect of the combination of rimonabant and phentermine on weight and cardiometabolic risk factors are studied in overweight and obese patients in a 2-year, randomized, double-blind, placebo-controlled trial. Individuals are assessed and pre-screened to assemble an experimental group of subjects which include men and women aged 18 years or older having a body mass index of 30 or greater (for the obesity study) or 27 or greater (for the overweight study including treated or untreated dyslipidemia or hypertension). Initial screening includes a medical history, physical examination, electrocardiography, clinical chemistry, thyroid function, hematology, and urinalysis. Body weight is measured using a calibrated digital or balance scale at screening, biweekly during the run-in period, baseline (randomization), weeks 2 and 4, and then every 4 weeks. Waist circumference is measured using a spring-loaded measuring tape midway between the lower rib and iliac crest and follow the same measurement schedule as body weight.

Patients are excluded if they have a body weight fluctuation of more than 5 kg in the previous 3 months; clinically significant cardiac, renal, hepatic, gastrointestinal tract, neuropsychiatric, or endocrine disorders; drug-treated or diagnosed type 1 or type 2 diabetes; use of medications that alter body weight or appetite; a history of substance abuse or current substance abuse; or changes in smoking habits or smoking cessation within the past 6 months.

Following a 1-week screening period, patients are instructed to follow a hypocaloric diet (approximately 600 kcal/d deficit) that is continued during a 4-week placebo, single-blind, run-in period and then throughout the double-blind treatment period. The diet prescription is adjusted to each patient's basal metabolic rate which is estimated by the Harris-benedict equation and self-reported physical activity at screening and at weeks 24, 52, and 76. Patients also are instructed to increase their level of physical activity throughout the study.

Patients who complete the run-in period are randomly allocated to 1 of 9 double-blind treatment groups: placebo, 5 mg/d of rimonabant alone, 10 mg/d of rimonabant alone, 15 mg/d of rimonabant alone, 20 mg/d of rimonabant alone, 5 mg/d of rimonabant and 15 mg/d of phentermine, 10 mg/d of rimonabant and 15 mg/d of phentermine, 15 mg/d of rimonabant and 15 mg/d of phentermine or 20 mg/d of rimonabant and 15 mg/d of phentermine.

Fasting serum glucose and insulin levels are measured at screening, baseline, every 12 weeks until week 36, at week 52, every 12 weeks between week 52 and week 88, and at week 104. Serum glucose, insulin, and lipids are assayed according to standard procedures. Low-density lipoprotein cholesterol are measured directly by ultracentrifugation. Metabolic syndrome status is assessed according to the National Cholesterol Education Program Expert Panel on Detection, Evaluation, and Treatment of High Blood Cholesterol in Adults (Adult Treatment Panel III) criteria at baseline, year 1, and year 2.

It is expected that the combination of rimonabant and phentermine will be as effective in reducing body weight and waist circumference as compared to the rimonabant doses administed alone while also favorably affecting several cardiometabolic risk factors. In addition, the combination of 5 mg/d of rimonabant and 15 mg/d of phentermine, will be just as effective in reducing body weight and affecting cardiometabolic risk factors as the 20 mg dose of rimonabant alone but will have a reduction in adverse side effects.

### EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby fully incorporated by reference. The appropriate components, processes, and methods of those patents, applications and other documents may be selected for the present invention and embodiments thereof.

1. A composition for treating obesity or a related condition in a subject comprising: a first pharmaceutical agent and a second pharmaceutical agent, wherein the second pharmaceutical agent is a sympathomimetic agent and wherein the first pharmaceutical agent is an anti-epileptic agent, CB1 receptor antagonist, or a 5HT_{2C}-selective serotonin receptor agonist.

2. The composition of claim 1, wherein said sympathomimetic agent is phentermine or bupropion.

3. The composition of claim 1, wherein said first pharmaceutical agent is an anti-epileptic agent.

4. The composition of claim 3, wherein said anti-epilpetic agent is selected from the group consisting of topiramate, zonisamide, γ-vinyl GABA (vigabatrin), carbamazepine, clonazepam, ethosuximide, gabapentin, lamotrigine, levetiracetam, phenobarbital, phenyloin, primidone, tiagabine, valproate, felbamate, oxazinane-dione, metharbital, ethotoin and mesantoin, vigabatrin, gabapentin, and oxcarbazepine.

5. The composition of claim 3, wherein said anti-epileptic agent is topiramate.

6. The composition of claim 3, wherein said anti-epileptic agent is zonisamide.

7. The composition of claim 1, wherein said first pharmaceutical agent is a CB1 receptor antagonist.

8. The composition of claim 7, wherein said CB1 receptor antagonist is selected from the group consisting of rimonabant, SLV-326, SLV-319, AM251, AM4113, AM281, Taranabant, NIDA-41020, NEWW 0327, O-2050, O-2654, CP-272871, CP-945598, CP-946598, AVE1625, Surinabant, LY320135, AVN-342, GRC-10389, Org-50189, PSNCBAM-1, E-6776, V-24343, ACPA, ACEA, HU-210, and HU-243.

9. The composition of claim 7, wherein said CB 1 receptor antagonist is rimonabant.

10. The composition of claim 1, wherein said first pharmaceutical agent is a 5HT_{2C}-selective serotonin receptor agonist.

11. The composition of claim 10, wherein said 5HT_{2C}-selective serotonin receptor agonist is selected from the group consisting of lorcaserin, mesulergine, agomelatine, fluoxetine, BVT933, DPCA37215, 1K264; PNU 22394; WAY161503, R-1065, and YM 348.

12. The composition of claim 10, wherein said 5HT_{2C}-selective serotonin receptor agonist is lorcaserin.

13. The composition of claim 1, comprising a dosage form comprising an immediate release form of the sympathomimetic agent and a controlled release form of the anti-epileptic agent, CB1 receptor antagonist, or 5HT_{2C}-selective serotonin receptor agonist.

14. The composition of claim 13, wherein the sympathomimetic agent is phentermine.

15. The composition of claim 13, wherein the sympathomimetic agent is bupropion.

16. The composition of claim 13, wherein the anti-epileptic agent is topiramate.

17. The composition of claim 13, wherein the anti-epileptic agent is zonisamide.

18. The composition of claim 13, wherein the CB1 receptor antagonist is rimonabant.

19. The composition of claim 13, wherein the 5HT_{2C}-selective serotonin receptor agonist is lorcaserin.

20. A method for treating obesity or a related condition in a subject comprising administering to the subject a first pharmaceutical agent and a second pharmaceutical agent, wherein the second pharmaceutical agent is a sympathomimetic agent and wherein the first pharmaceutical agent is an anti-epileptic agent, CB1 receptor antagonist, or a 5HT_{2C}-selective serotonin receptor agonist.

21. The method of claim 20, wherein the first pharmaceutical agent and the second pharmaceutical agent are administered separately.

22. The method of claim 21, wherein the first pharmaceutical agent and the second pharmaceutical agent are administered at different times of the day.

23. The method of claim 22, wherein the second pharmaceutical agent is administered in the morning and the first pharmaceutical agent is administered at least once later in the day.

24. The method of claim 23, wherein the second pharmaceutical agent is phentermine.

25. The method of claim 24, wherein the phentermine is an immediate release dosage form.

26. The method of claim 25, wherein the first pharmaceutical agent is topiramate.

27. The method of claim 25, wherein the first pharmaceutical agent is zonisamide.

28. The method of claim 25, wherein the first pharmaceutical agent is rimonabant.

29. The method of claim 25, wherein the first pharmaceutical agent is lorcaserin.

30. The method of claim 23, wherein the second pharmaceutical agent is bupropion.

31. The method of claim 30, wherein the bupropion is an immediate release dosage form.

32. The method of claim 31, wherein the first pharmaceutical agent is topiramate.

33. The method of claim 31, wherein the first pharmaceutical agent is zonisamide.

34. The method of claim 31, wherein the first pharmaceutical agent is rimonabant.

35. The method of claim 31, wherein the first pharmaceutical agent is lorcaserin.

36. The method of claim 20, wherein the condition is pre-diabetes, insulin-resistance or diabetes.

37. The method of claim 20, wherein the condition is hypertension.

38. The method of claim 20, wherein the condition is sleep apnea.

39. The method of claim 20, wherein the condition is nonalcoholic steatohepatitis.

40. The method of claim 20, wherein the condition is nonalcoholic fatty liver disease.

41. The method of claim 20, wherein the condition is diabetic nephropathy.

42. A kit comprising a packaged combination of a first pharmaceutical agent and a second pharmaceutical agent, wherein the second pharmaceutical agent is a sympathomimetic agent and wherein the first pharmaceutical agent is an anti-epileptic agent, CB1 receptor antagonist, or a 5HT_{2C}-selective serotonin receptor agonist and instructions for a patient to carry out drug administration to achieve weight loss, wherein the first and second pharmaceutical agents are present in separate and discrete dosage forms.

43. A kit comprising a sealed package of controlled release dosage forms each containing a first pharmaceutical agent and a second pharmaceutical agent, wherein the second pharmaceutical agent is a sympathomimetic agent and wherein the first pharmaceutical agent is an anti-epileptic agent, CB1 receptor antagonist, or a 5HT_{2C}-selective serotonin receptor agonist, wherein the dosage forms provide for immediate release of the second pharmaceutical agent and delayed release of the first pharmaceutical agent.
